(19) United States
(12) Patent Application Publication
(10) Pub. No.: US 2023/0337368 A1
(43) Pub. Date: Oct. 19, 2023

(54) METHOD FOR PREPARING SCREEN-PRINTED BIOELECTROCHEMICAL SENSOR

(71) Applicants: Shaoda ZHANG, Guangdong (CN); Xingyu Zheng, Guangdong (CN); Jinbao Wang, Guangdong (CN)

(72) Inventors: Shaoda ZHANG, Guangdong (CN); Xingyu Zheng, Guangdong (CN); Jinbao Wang, Guangdong (CN)

(21) Appl. No.: 17/946,032

(22) Filed: Sep. 16, 2022

(30) Foreign Application Priority Data
Apr. 14, 2022 (CN) 202210387869.0

(51) Int. Cl. H05K 3/12; C12Q 1/00; G01N 27/30

(52) U.S. Cl. CPC H05K 3/1216; C12Q 1/006; G01N 27/308

(57) ABSTRACT

The present disclosure discloses a method for preparing a screen-printed bioelectrochemical sensor. A conductive polymer polyaniline is deposited on a surface of a screen-printed carbon electrode by electropolymerization. The conductive polymer has strong adsorption property, and is used for immobilization of glucose oxidase, so that the long-term stability of the glucose oxidase on the carbon electrode is improved. Meanwhile, a polyethylene glycol glycidyl ether cross-linked polymer ferrocene and the glucose oxidase are used, so that the immobilization of the glucose oxidase is further strengthened. Moreover, a hydrogel outer membrane with a cross-linking effect is used, so that the stability of an electrode product is further improved, and great linearity is maintained.

### BACKGROUND OF THE INVENTION

The present disclosure relates to the technical field of bioelectrochemical sensors, and more specifically, relates to a method for preparing a screen-printed bioelectrochemical sensor.

Diabetes is a metabolic disorder syndrome caused by various pathogenic factors such as genetic factors and immune factors acting on the body, leading to hypoinsulinism and insulin resistance. Thus, it is very necessary to detect blood glucose of diabetic patients during treatment. Bioelectrochemical sensors have simplicity, convenience, low price, high sensitivity, and other advantages. Therefore, the bioelectrochemical sensors have been widely used in medical and health treatment, and play a major role in detection of the blood glucose of the diabetic patients. The blood glucose is monitored by using the bioelectrochemical sensors, usually glucose bioelectrochemical sensors. The glucose bioelectrochemical sensors have various test principles and methods, including an oxidase method, a spectral analysis method, and a fluorescence detection method. At present, a glucose oxidase method is the most mature technology with highest detection precision. According to the method, the concentration of the blood glucose is measured by using an electrochemical method based on a current change during a glucose reaction catalyzed by glucose oxidase. A first glucose enzyme electrode is prepared by embedding a thin layer of glucose oxidase into an oxygen electrode through a semipermeable membrane, and the consumption amount of oxygen in an enzyme catalytic process can be measured. Since then, many researchers have focused on research and development of glucose electrochemical sensors, and have made great progress.

According to catalytic properties of the glucose oxidase, the development of the glucose bioelectrochemical sensors can be divided into three stages including first-generation sensors using oxygen as an electron acceptor, second-generation sensors using non-physiological mediators as electron acceptors, and third-generation sensors for direct electron transfer. The first-generation glucose bioelectrochemical sensors have the disadvantage of high test voltage, and the problem of over-reliance on oxygen in high concentration of glucose, so that the linearity of the sensors is affected. In order to reduce the working potential of the sensors, reduce the influence of interfering substances on currents of the sensors, and solve the problem of the influence of insufficient oxygen on the linearity of the sensors, artificial electronic mediators are used for replacing oxygen to serve as electronic media in the second-generation glucose bioelectrochemical sensors. In order to achieve functions of electron mediators, a reaction rate of the artificial electron mediators and the glucose oxidase is required to be much greater than that of the oxygen and the glucose oxidase. In this cause, effects of the oxygen are minimized. Such artificial electron mediators include ferrocene, ferricyanide, conductive organic salts, transition metal complexes, and the like. These substances have low redox voltage, so that the working voltage of the sensors can be reduced, and the influence of the interfering substances can be eliminated in a low voltage state of the sensors.

After the glucose bioelectrochemical sensors are implanted subcutaneously, the sensitivity is gradually reduced. On the one hand, this phenomenon is caused by an allogeneic reaction. On the other hand, the activity of the glucose oxidase is constantly reduced. An enzyme immobilization technique for preparation of the bioelectrochemical sensors is involved. In the prior art, substances such as glutaraldehyde and genipin are used for immobilization of the glucose oxidase. These substances not only have high toxicity and serious pollution, but also have a certain influence on the activity of the glucose oxidase during immobilization of the glucose oxidase. When the glucose bioelectrochemical sensors are implanted subcutaneously, the sensitivity is greatly reduced under the action of the allogeneic reaction, and finally, detection results of the glucose bioelectrochemical sensors are affected.

### BRIEF SUMMARY OF THE INVENTION

Existing glucose biosensors have low load amount of glucose oxidase and low stability. When a glucose biosensor is implanted subcutaneously in a human body, the activity of the glucose oxidase attached to a sensing electrode is constantly reduced due to an allogeneic reaction, and detection results of the glucose bioelectrochemical sensor are seriously affected. In order to overcome the defects of the prior art, the present disclosure provides a method for preparing a screen-printed bioelectrochemical sensor.

The present disclosure adopts the following technical solutions.

A method for preparing a screen-printed bioelectrochemical sensor comprises the following process steps:
step S1, preparing a screen-printed carbon electrode, and cleaning a surface of the screen-printed carbon electrode;
step S2, preparing an aniline solution, and putting the screen-printed carbon electrode in the aniline solution for an electropolymerization reaction by using a galvanostatic method;
step S3, preparing a mixed enzyme solution, and putting the screen-printed carbon electrode in the mixed enzyme solution for standing;
step S4, soaking the screen-printed carbon electrode in deionized water; and
step S5, preparing an outer membrane material solution, and soaking the screen-printed carbon electrode in the outer membrane material solution.

According to the above-mentioned method for preparing a screen-printed bioelectrochemical sensor, in step S1, a cleaning process comprises:
step A1, cleaning the screen-printed carbon electrode for 3 minutes by using an ultrasonic cleaner;
step A2, drying the cleaned screen-printed carbon electrode in an environment at 30 t;
step A3, cleaning the screen-printed carbon electrode for 180 seconds by using a plasma cleaner; and
step A4, cleaning a surface of the screen-printed carbon electrode for 30 minutes by using a chemical workstation based on chronoamperometry.

According to the above-mentioned method for preparing a screen-printed bioelectrochemical sensor, in step S2, aniline is put in a 0.2 mmol/l HCl solution to form a 0.4 mmol/l aniline solution.

According to the above-mentioned method for preparing a screen-printed bioelectrochemical sensor, in step S2, electropolymerization is performed at a constant current of 0.1 mA for 10 minutes.

According to the above-mentioned method for preparing a screen-printed bioelectrochemical sensor, in step S3, a process of preparing the mixed enzyme solution comprises:
step B1, adding a saturated solution of a ferrocene polymer with a pH of 5.5 to a reaction vessel;
step B2, adding 20 mg/L of glucose oxidase to the reaction vessel;
step B3, adding 40% of polyethylene glycol glycidyl ether to the reaction vessel; and
step B4, performing standing for 24 hours.

Further, a mass ratio of the saturated solution of a ferrocene polymer added to the reaction vessel to the glucose oxidase to the polyethylene glycol glycidyl ether is 140:60:75.

According to the above-mentioned method for preparing a screen-printed bioelectrochemical sensor, in step S3, the screen-printed carbon electrode is soaked in the mixed enzyme solution for standing for 2 hours.

According to the above-mentioned method for preparing a screen-printed bioelectrochemical sensor, in step S4, the screen-printed carbon electrode is soaked in the deionized water for 2 hours, and then dried in an oven at 25° C.

According to the above-mentioned method for preparing a screen-printed bioelectrochemical sensor, in step S5, the outer membrane material solution is a PSS solution, and a solution with a mass percentage of 3% prepared from 98% of deionized water and 2% of dimethylformamide is used as a solvent.

According to the above-mentioned solutions, the present disclosure has the following beneficial effects. In the present disclosure, a conductive polymer polyaniline is deposited on a surface of a screen-printed carbon electrode by electropolymerization. The conductive polymer has strong adsorption property, and is used for immobilization of glucose oxidase, so that the long-term stability of the glucose oxidase on the carbon electrode is improved. Meanwhile, a polyethylene glycol glycidyl ether cross-linked polymer ferrocene and the glucose oxidase are used, so that the immobilization of the glucose oxidase is further strengthened. Moreover, a hydrogel outer membrane with a cross-linking effect is used, so that the stability of an electrode product is further improved, and great linearity is maintained.
1. Compared with cross-linking methods using a glutaraldehyde solution and genipin, or other preparation methods such as embedding and sol-gel in the prior art, the preparation method in the present disclosure has a better enzyme immobilization effect. In the prior art, organic substances such as glutaraldehyde are used for cross-linking, so that the enzyme activity is greatly influenced. However, in the present disclosure, an enzyme and an electron mediator are immobilized, so that efficient transmission of electrons is ensured, the current density of the electrochemical sensor can reach 118 nA/mm2, and the stability is also better than that of an electrode prepared in the prior art. According to an experimental test structure, the activity of an electrode prepared by using the preparation method of the present disclosure can reach 60,000 U after 1 year.
2. In the present disclosure, the polyaniline is deposited on the surface of the electrode by electropolymerization, so that not only can the adsorption capacity of the enzyme be strengthened, the stability of the enzyme immobilized on the surface of the electrode is improved, but also an effect of improving the transmission rate of electrons is achieved.
3. In the present disclosure, the polyethylene glycol glycidyl ether is used as a cross-linking agent for the enzyme, and has a better cross-linking effect than other cross-linking agents.
4. The glucose bioelectrochemical sensor prepared by immobilizing the polyaniline on a screen-printed carbon substrate by electropolymerization in the present disclosure has a linear coefficient of greater than 0.98 within a confidence interval of 95%, a linear range of 1.7-28 mmol/I, and high stability. The stability can be constantly maintained for more than 40 days, and the current signal is attenuated by about 60%. Signals generated by the electrochemical sensor after 4 days in a test process are basically unchanged, and the stability is greatly improved in comparison with that of an electrochemical sensor prepared in the prior art.

### DETAILED DESCRIPTION OF THE INVENTION

In order to make the technical problems to be solved, technical solutions and beneficial effects of the present disclosure clearer, the present disclosure is further described in detail below with reference to the accompanying drawings and embodiments. It should be understood that the specific embodiments described herein are only for illustrating but not for limiting the present disclosure.

A method for preparing a screen-printed bioelectrochemical sensor includes the following process steps.

Step S1, a screen-printed carbon electrode is prepared, and a surface of the screen-printed carbon electrode is cleaned. In this process, since an oxidase needs to be immobilized on the surface of the screen-printed carbon electrode, the surface of the screen-printed carbon electrode is required to be cleaned to remove other impurities, dirt, and the like on the surface, so that the deposition of a polymer and the attachment of the oxidase in subsequent processes are prevented from being contaminated or interfered. In the present disclosure, the screen-printed carbon electrode is used as an electrode of the bioelectrochemical sensor, and a working electrode, a counter electrode, and a reference electrode are all prepared from bio-carbon materials. The electrodes are separated by insulating layers to prevent a short circuit of the electrodes, and the screen-printed electrode is more delicate, smaller in size, and lower in material cost.

In this embodiment, in step S1, a cleaning process includes:
step A1, cleaning the screen-printed carbon electrode for 3 minutes by using an ultrasonic cleaner;
step A2, drying the cleaned screen-printed carbon electrode in an environment at 30° C.;
step A3, cleaning the screen-printed carbon electrode for 180 seconds by using a plasma cleaner; and
step A4, cleaning a surface of the screen-printed carbon electrode for 30 minutes by using a chemical workstation based on chronoamperometry.

After steps A1 to A3, cross-linking agent particles and oil stains on the surface of the screen-printed carbon electrode are removed, and then the screen-printed carbon electrode is cleaned by using a chemical workstation based on chronoamperometry to achieve the effect of activating the surface of the screen-printed carbon electrode.

Step S2, an aniline solution is prepared, and the screen-printed carbon electrode is put in the aniline solution for an electropolymerization reaction by using a galvanostatic method. The current magnitude and time for polymerization are determined by the uniformity of electropolymerization and the particle size of molecules. During the electropolymerization reaction, due to stable current and voltage in the galvanostatic method, stable, uniform, and dense polyaniline can be formed on the surface of the screen-printed carbon electrode in the aniline solution.

Due to non-electrolytic enzyme, low electrolysis efficiency, and low load amount of the enzyme, glucose oxidase is only used to cross-link with an amino group of the polyaniline. The glucose oxidase has low stability and slow transmission of electrons. Meanwhile, since a by-product hydrogen peroxide produced by glucose under catalysis of the glucose oxidase also has a certain influence on the enzyme activity, in this application, aniline is electropolymerized on the surface of the screen-printed carbon electrode by using the galvanostatic method, so that a group that can bind to the glucose oxidase, an electron mediator, and a cross-linking agent is formed on the surface of the screen-printed carbon electrode, and the stability of the glucose oxidase on the screen-printed carbon electrode is further improved without affecting transmission of electrons during sensing.

In step S2, aniline is put in a 0.2 mmol/l HCl solution to form a 0.4 mmol/l aniline solution.

In an embodiment, 3.72 g of an aniline monomer is dissolved in 100 ml of a 0.2 mmol/l solution to form a 0.4 mmol/ml aniline solution.

The screen-printed carbon electrode is put in the aniline solution for electropolymerization by using the galvanostatic method at a current of 0.1 mA. After the screen-printed carbon electrode is cleaned with deionized water, it can be found that the surface of the screen-printed carbon electrode is dark green, due to the growth of the polyaniline on the surface of the screen-printed carbon electrode. At this moment, a polyaniline network is attached to the surface of the screen-printed carbon electrode.

Step S3, a mixed enzyme solution is prepared, and the screen-printed carbon electrode is put in the mixed enzyme solution for standing.

In this application, the glucose oxidase is adsorbed in the polyaniline network on the surface of the screen-printed carbon electrode by electrostatic interaction. Due to a low electrostatic force, the glucose oxidase is likely to leak and overflow, so that a reaction between the electrode and a detection solution is affected. Therefore, a cross-linking agent is required for cross-linking.

In this embodiment, in step S3, a process of preparing the mixed enzyme solution includes:
step B1, adding a saturated solution of a ferrocene polymer with a pH of 5.5 to a reaction vessel;
step B2, adding 20 mg/L of glucose oxidase to the reaction vessel;
step B3, adding 40% of polyethylene glycol glycidyl ether to the reaction vessel; and
step B4, performing standing for 24 hours.

In this embodiment, a mass ratio of the saturated solution of a ferrocene polymer added to the reaction vessel to the glucose oxidase to the polyethylene glycol glycidyl ether is 140:60:75.

In this embodiment, in step S3, the screen-printed carbon electrode is soaked in the mixed enzyme solution for standing for 2 hours.

According to the above-mentioned steps, the saturated solution of a ferrocene polymer, the glucose oxidase, and the polyethylene glycol glycidyl ether are sequentially added to the reaction vessel for a cross-linking reaction of the glucose oxidase for 24 hours, and then the screen-printed electrode is soaked in the mixed enzyme solution for standing at room temperature for 2 hours, so that the cross-linked glucose oxidase in the mixed enzyme solution can fully enter the polyaniline nanofiber network on the surface of the screen-printed electrode. The glucose oxidase is adsorbed in the polyaniline network by electrostatic interaction. 2 hours later, the screen-printed electrode is taken out, and then dried in an oven at 25° C.

In this application, the polyethylene glycol glycidyl ether is used as a cross-linking agent. As an alcohol polymer, the polyethylene glycol glycidyl ether has little influence on the activity of the glucose oxidase, and the enzyme activity is not affected even after a polymerization reaction in the solution for a long time. Meanwhile, the ferrocene polymer and the oxidase are cross-linked by using the cross-linking agent based on a terminal-hydroxyl group, and a cross-linking reaction of the three substances is a competitive reaction. When the content of the glucose oxidase is too high, an excessive reaction of the cross-linking agent and the glucose oxidase may be caused, less electron mediators are involved in the reaction, and as a result, rapid transfer of electrons cannot be achieved. When the content of the glucose oxidase is too low, the catalytic strength for glucose may be low, and the current signal is low. Since the three substances are polymers with long molecular chains, the required reaction time is long. When the reaction time is short, the glucose oxidase may be immobilized unstably, and the response time of an electrical signal is short. When the reaction time is too long, excessive cross-linking is caused, a large number of glucose oxidase active sites are occupied by the cross-linking agent, and the enzyme activity is reduced.

Step S4, the screen-printed carbon electrode is soaked in deionized water. In the process of attaching the glucose oxidase to the polyaniline network, a part with low adhesion strength exists. The screen-printed carbon electrode is soaked in the deionized water for a long time for allowing these unimmobilized glucose oxidase and other electron mediators to be diffused into water, so that the glucose oxidase firmly immobilized on the surface of the screen-printed electrode is ensured, and the stability of the electrochemical sensor is ensured.

In this embodiment, in step S4, the screen-printed carbon electrode is soaked in the deionized water for 2 hours, and then dried in an oven at 25° C.

Step S5, an outer membrane material solution is prepared, and the screen-printed carbon electrode is soaked in the outer membrane material solution. In order to improve the linearity and stability of the sensor, the glucose oxidase firmly attached to the surface of the screen-printed electrode is further immobilized, and a PSS outer membrane is attached to the outermost layer of the screen-printed carbon electrode. The PSS outer membrane is used for coating the glucose oxidase to achieve the effects of protection and immobilization, and the stability of the bioelectrochemical sensor is improved without affecting a current sensing effect. The screen-printed carbon electrode is soaked in the outer membrane material solution for 3 minutes, taken out, and naturally dried at room temperature for spare use. Then, the screen-printed carbon electrode can be tested.

In this embodiment, in step S5, the outer membrane material solution is a PSS solution, and a solution with a mass percentage of 3% prepared from 98% of deionized water and 2% of dimethylformamide is used as a solvent.

The foregoing descriptions are only preferred embodiments of the present disclosure, and are not intended to limit the present disclosure. Any modifications, equivalent substitutions, improvements and the like made within the spirit and principle of the present disclosure shall be included within the protection range of the present disclosure.

What is claimed is:

1. A method for preparing a screen-printed bioelectrochemical sensor, comprising the following process steps:
step S1, preparing a screen-printed carbon electrode, and cleaning a surface of the screen-printed carbon electrode;
step S2, preparing an aniline solution, and putting the screen-printed carbon electrode in the aniline solution for an electropolymerization reaction by using a galvanostatic method;
step S3, preparing a mixed enzyme solution, and putting the screen-printed carbon electrode in the mixed enzyme solution for standing;
step S4, soaking the screen-printed carbon electrode in deionized water; and
step S5, preparing an outer membrane material solution, and soaking the screen-printed carbon electrode in the outer membrane material solution.

2. The method for preparing a screen-printed bioelectrochemical sensor according to claim 1, wherein in step S1, a cleaning process comprises:
step A1, cleaning the screen-printed carbon electrode for 3 minutes by using an ultrasonic cleaner;
step A2, drying the cleaned screen-printed carbon electrode in an environment at 30° C.;
step A3, cleaning the screen-printed carbon electrode for 180 seconds by using a plasma cleaner; and
step A4, cleaning a surface of the screen-printed carbon electrode for 30 minutes by using a chemical workstation based on chronoamperometry.

3. The method for preparing a screen-printed bioelectrochemical sensor according to claim 1, wherein in step S2, aniline is put in a 0.2 mmol/l HCl solution to form a 0.4 mmol/l aniline solution.

4. The method for preparing a screen-printed bioelectrochemical sensor according to claim 1, wherein in step S2, electropolymerization is performed at a constant current of 0.1 mA for 10 minutes.

5. The method for preparing a screen-printed bioelectrochemical sensor according to claim 1, wherein in step S3, a process of preparing the mixed enzyme solution comprises:
step B1, adding a saturated solution of a ferrocene polymer with a pH of 5.5 to a reaction vessel;
step B2, adding 20 mg/L of glucose oxidase to the reaction vessel;
step B3, adding 40% of polyethylene glycol glycidyl ether to the reaction vessel; and
step B4, performing standing for 24 hours.

6. The method for preparing a screen-printed bioelectrochemical sensor according to claim 5, wherein a mass ratio of the saturated solution of a ferrocene polymer added to the reaction vessel to the glucose oxidase to the polyethylene glycol glycidyl ether is 140:60:75.

7. The method for preparing a screen-printed bioelectrochemical sensor according to claim 1, wherein in step S3, the screen-printed carbon electrode is soaked in the mixed enzyme solution for standing for 2 hours.

8. The method for preparing a screen-printed bioelectrochemical sensor according to claim 1, wherein in step S4, the screen-printed carbon electrode is soaked in the deionized water for 2 hours, and then dried in an oven at 25° C.

9. The method for preparing a screen-printed bioelectrochemical sensor according to claim 1, wherein in step S5, the outer membrane material solution is a PSS solution, and a solution with a mass percentage of 3% prepared from 98% of deionized water and 2% of dimethylformamide is used as a solvent.
